(19) United States
(12) Patent Application Publication
(10) Pub. No.: US 2017/0209081 A1
(43) Pub. Date: Jul. 27, 2017

(54) DEVICE, SYSTEM AND METHOD FOR NON-INVASIVELY MEASURING BLOOD GLUCOSE

(71) Applicant: Biolab Technologies Ltd., Jerusalem (IL)

(72) Inventor: Ohad Samuel Davidson, Rosh HaAyin (IL)

(73) Assignee: Biolab Technologies Ltd., Jerusalem (IL)

(21) Appl. No.: 15/328,163

(22) PCT Filed: Aug. 13, 2015
(86) PCT No.: PCT/IB15/56162

§ 371 Date: Jan. 23, 2017

Related U.S. Application Data
(60) Provisional application No. 62/039,047, filed on Aug. 19, 2014.

(51) Int. Cl. A61B 5/145; A61B 5/00; A61B 5/1455

(52) U.S. Cl. CPC A61B 5/14532; A61B 5/1455; A61B 5/6826; A61B 5/6816

(57) ABSTRACT

Device and method for non-invasively measuring the level of glucose in blood. The device comprises a light shielded chamber, a light emitter operative to emit light in the green-turquoise (GT) wavelength range into the light shielded chamber, a detector operative to detect GT light in the GT wavelength range in the light shielded chamber, a tissue receiving area for removably receiving at least a part of a human tissue such to be housed within the light shielded chamber so that the emitted light is incident onto the human tissue, and a monitoring engine operative to determine a level of glucose in blood based on the amount of emitted and detected GT light.

### CROSS REFERENCE TO RELATED APPLICATIONS

This application is a 371 application from international patent application PCT/IB2015/056162 having the same title, and is related to and claims priority from U.S. Provisional Patent Application No. 62/039,047 titled “NON-INVASIVE BLOOD GLUCOSE MONITORING”, which is incorporated herein by reference in its entirety.

### TECHNICAL FIELD

The present patent application relates to methods and devices for monitoring a human physiological parameter. More specifically, embodiments disclosed herein relate to monitoring of blood glucose in humans.

### BACKGROUND

Diabetes is a world epidemic health problem that is expected to increase with the aging of the population everywhere. Self-monitoring of blood glucose (“SMBG”) levels is a significant component of diabetic patient care. SMBG works by having patients perform a number of glucose tests each day or each week. The test used most commonly involves pricking a finger with a lancet device to obtain a small blood sample, applying a drop of blood onto a reagent strip, and determining the glucose concentration by inserting the strip into a reflectance photometer for an automated reading. Test results are then recorded in a logbook or stored in a glucose meter's electronic memory.

However, it is well recognized that the vast majority of diabetes patients, particularly Type II diabetes patients, still do not test nearly as frequently as they should, and some still do not test at all.

The description above is presented as a general overview of related art in this field and should not be construed as an admission that any of the information it contains constitutes prior art against the present patent application.

### SUMMARY

Aspects of disclosed embodiments provide for a non-invasive glucose measuring and monitoring (GM) device that includes a mechanism for measuring green-turquoise (GT) light in the range of for example about 490 nm to 505 nm.

Disclosed embodiments provide for a non-invasive glucose measuring and monitoring device including a light shieldable chamber with an optic reader having an LED (light emitting diode) or laser source module with a mechanism for measuring GT light, a photodiode module sensitive to light spectrum ranging from UV (e.g., about 350 nm-400 nm) to IR (e.g., about 1100 nm), a capacitance sensor for detecting contact and/or measuring pressure (absolute or relative pressure, e.g., relative to atmospheric pressure) applied by the human finger and a test pad that attaches to the human tissue. It is noted that the IR range may also encompass the near-infrared or NIR wavelength range.

Disclosed embodiments provide for a method for measuring blood glucose in an individual by measuring absorbance of GT light, and for determining a blood glucose level based on the amount of GT light absorbed. To clarify, “absorbed light” is emitted minus detected light.

It is noted that the terms “determining” and “measuring” as well as grammatical variations thereof may be used interchangeably.

Example 1 includes a device for non-invasively measuring the level of glucose in blood, the device comprising: a light shielded chamber; a light emitter operative to emit light in the green-turquoise wavelength range into the light shielded chamber; a detector operative to detect light in the green-turquoise wavelength range in the light shielded chamber; a tissue receiving area for removably receiving at least a part of a human tissue such to be housed within the light shielded chamber so that the emitted light is incident onto the human tissue; and a monitoring engine operative to determine a level of glucose in blood based on the amount of emitted and detected light.

Example 2 includes the subject matter of example 1 and, optionally, wherein the emitted light has a wavelength ranging from 490 nm to 505 nm.

Example 3 includes the subject matter of examples 1 or 2 and, optionally, further comprising an IR light emitter and detector for determining one or both of pressure applied onto the human tissue and a time stamp of a maximum blood pulse value in the human tissue.

Example 4 includes the subject matter of any of the examples 1-3 and, optionally, further comprising a capacitance sensor for detecting whether contact is made with the sensor receiving area and/or for determining the pressure applied by the human tissue portion onto the sensor receiving area.

Example 5 includes the subject matter of any of the examples 1-4 and, optionally, wherein at least one of the light emitter and detector are mounted onto a mechanical energy storage device such that light emitter and/or detector may be pressed by the mechanical energy storage device against the human tissue portion received by the tissue receiving area.

Example 6 includes the subject matter of any of the examples 1-5 and, optionally, wherein a correlation between at least 5 Oral Glucose Tolerance Test (OGTT) blood glucose measurements performed by the device and the measurements performed using a Freestyle Freedom-Lite® device is at least 0.8 with a statistical significance of P<0.0001.

Example 7 includes the subject matter of any of the examples 1-6 and, optionally, wherein detection of the amount of GT light is based on transmittance.

Example 8 includes the subject matter of any of the examples 1-7 and, optionally, wherein detection of the amount of GT light is based on reflectance.

Example 9 includes a method for non-invasively measuring the level of glucose in blood, the method comprising: emitting light in the GT wavelength towards a human tissue portion; detecting an attenuated amount of the emitted light which propagated through the human tissue; and determining, based on the emitted light and the detected attenuated amount of emitted light, a level of glucose in blood in the human tissue.

Example 10 includes the subject matter of example 9 and, optionally, comprising emitting the light at a wavelength ranging from 490 nm to 505 nm.

Example 11 includes the subject matter of example 9 or 10 and, optionally, further comprising determining one or both of pressure applied onto the human tissue and a time stamp of a maximum blood pulse value in the human tissue, by employing an infrared (IR) light emitter and detector.

Example 12 includes the subject matter of any of examples 9-11 and, optionally, further comprising detecting whether contact is made with the sensor receiving area and/or determining the pressure applied by the human tissue portion onto the sensor receiving area.

Example 13 includes the subject matter of example 12 and, optionally, wherein the detection of contact and/or determining the amount of applied pressure is accomplished by employing a capacitance sensor.

Example 14 includes the subject matter of any of examples 9-13 and, optionally, wherein detection of the amount of GT light is based on transmittance.

Example 15 includes the subject matter of any of examples 9-15 and, optionally, wherein detection of the amount of GT light is based on reflectance.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### DESCRIPTION OF THE DRAWINGS

Other advantages of disclosed embodiments are readily appreciated as the same becomes better understood by reference to the following detailed description when considered in connection with the accompanying drawings wherein:

FIG. 1A is a schematic illustration of an exemplary blood glucose measurement and monitoring (GM) device, according to an embodiment;

FIG. 1B is a schematic block diagram illustration of the exemplary GM device, according to an embodiment;

FIG. 1C is a schematic block diagram illustration of a monitoring engine of the exemplary GM device, according to an embodiment;

FIG. 2A is a schematic isometric top view illustration of a GM device in an open configuration showing schematically a body portion operably engaging with the GM device, according to an embodiment;

FIG. 2B is a schematic isometric top view illustration of the exemplary GM device in the open configuration with the body portion removed, according to an embodiment;

FIG. 2C is another schematic isometric top view illustration of the exemplary GM device, according to an embodiment;

FIG. 2D is a schematic isometric view illustration of the exemplary GM device in the closed configuration, according to an embodiment;

FIG. 3 is a flow chart illustration of a method for measuring glucose level in blood;

FIG. 4A shows Oral Glucose Tolerance Test (OGTT) plots as measured with the GM device;

FIG. 4B shows Oral Glucose Tolerance Test (OGTT) plots as measured with the Freestyle® glucose measurement device.

### DETAILED DESCRIPTION

Aspects of the disclosed embodiments are directed to a non-invasive glucose measuring and monitoring device, which may herein be referred to as GM device.

It is noted that terms “blood sugar”, “sugar in blood”, and “glucose” may herein be used interchangeably.

The GM device measures glucose level in blood based on spectroscopic absorbance measurements, e.g., in a transmittance mode. The GM device comprises an emitter that is operative to emit light in a green-turquoise wavelength range (e.g., about 490 nm-505 nm), and a detector which is operative to detect at least the same wavelength range such as, for example, 350 nm-505 nm. Moreover, the detector may be operative to detect light in the IR range (e.g., 1100 nm). Based on a measured absorption of light in the mentioned range propagating through human tissue, the GM device may determine a level of glucose in blood. Increased absorption may be considered to be indicative of a correspondingly higher glucose concentration level in blood. Conversely, decreased absorption may be indicative of a correspondingly lower glucose concentration level in blood. Accordingly, absorption of light in human tissue may positively correlate with the level of glucose in blood.

It is noted that terms “glucose level”, “glucose concentration”, and “glucose concentration level” may be used interchangeably.

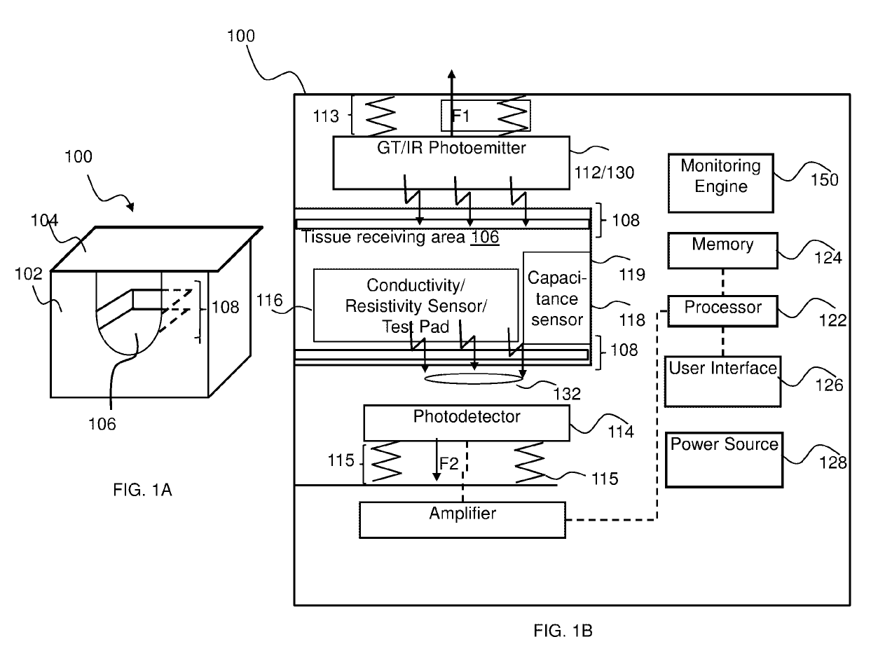

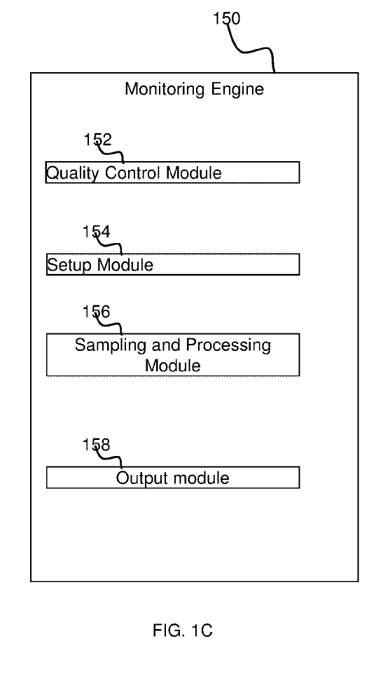

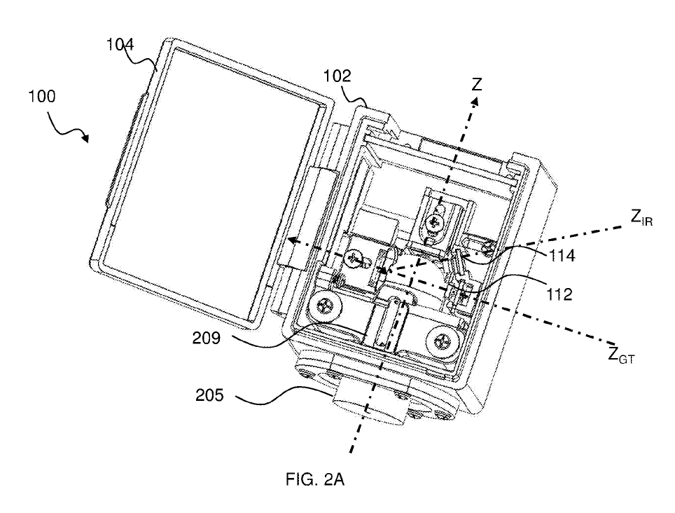

Reference is now made to FIG. 1A. A GM device 100 may include a light-shielded housing 102 comprising a cover 104 movable between open and closed positions. GM device 100 may further include a tissue receiving area 106, which may for example be a space or cavity sized such as to be able to removably receive at least part of a human finger (not shown) to be housed within light-shielded housing 102. Opening cover 104 allows tissue receiving area 106 to receive at least part of a human finger for example. After receipt of part of a human finger tissue receiving area 106 may be shielded from light by moving cover 104 into the closed position, shown in FIG. 1A.

Further referring to FIG. 1B, GM device 100 may comprise a GT photoemitter (or simply “emitter”) 112 and a photodetector 114. GT emitter 112 is operative to emit light in the GT wavelength range and may include, for example, a laser emitter module or a light emitting diode (LED).

It is noted that green-turquoise wavelength may range, for example, from about 485 nm-510 nm, 490 nm-505 nm, 490 nm-500 nm, 495 nm-505 nm or 495 nm-500 nm.

The absorption of green-turquoise light in the blood may correlate with glucose level in blood. Ranges of wavelength of GT light for determining glucose level in blood, based on absorption of GT light in blood, may include, for example, 492 nm to 498 nm, 490 nm to 505 nm, 490 nm to 500 nm (narrow range) and/or 485 nm to 510 nm. In an embodiment, absorption of blood may be optimal or relatively more responsive at wavelengths ranging from 492 nm to 498 nm. Light emitted by GT emitter 112 may comprise a plurality of wavelengths or a single wavelength within the GT wavelength ranges exemplified herein.

In an embodiment, photodetector 114 may be operative to detect light at a spectral range from about the UV range (e.g., about 350 nm-400 nm) to the infrared or IR range (e.g., about 1100 nm). Accordingly, photodetector 114 may be operative to detect light emitted by GT emitter 112. GT emitter 112 and photodetector 114 may be positioned relative to each other so that tissue receiving area 106 is located between GT emitter 112 and photodetector 114. More specifically, GT emitter 112 and photodetector 114 may be positioned relative to tissue receiving area 106 so that light emitted by GT emitter 112 propagates through a tissue region of the finger operably placed in or on tissue receiving area 106.

In an embodiment, applying pressure onto human tissue (e.g., by abutting GT emitter 112 and/or photodetector 114 against a portion of a finger, as outlined herein below in more detail) may result in acceleration of blood flow in the tissue where pressure was applied, yielding more blood in the tissue and thus increasing the ability to record differences in absorption of GT light by the blood. It is noted that the terms “absorption” and “attenuation” may be used interchangeably, both referring to a reduction in the intensity of photon flux.

The GT light may penetrate human tissue to a depth of, e.g., about 10 mm, when the LED/laser source is in contact with the skin. In an embodiment, the human tissue portion may be located with respect to the 112 emitter so that light emitted thereby is incident to the area of the location of a finger's distal phalanx. In an embodiment, the emitted light may be first incident onto the fingernail of a finger. In an embodiment, the emitted light is incident about in the center of a fingernail or nail plate.

Tissue receiving area 106 may receive at least a portion of the finger's distal phalanx so that it is sandwiched or positioned between GT emitter 112 and photodetector 114 to allow light emitted by the GT emitter to be incident onto tissue of the finger's distal phalanx and, eventually, propagate through tissue of the distal phalanx.

In an embodiment, tissue receiving area 106 may include a test pad 116 where the user positions a portion of his/her finger or other body part (e.g. an ear lobe) for allowing GT light to pass through the body part for measuring the level of glucose level in blood. To ensure propagation of light through tissue, substantially all of test pad 116 may have to be covered by the body or tissue portion received by tissue receiving area 106. In an embodiment, test pad 116 may be located in device 100 in any area that can easily test the human tissue. Test pad 116 may be made from any suitable material including, for example, plastic, rubber, or metals.

In an embodiment, test pad 116 may comprise a conductivity/electrical resistance sensor which engages with the tissue when the body portion (such as a fingertip or ear lobe) is set in an operable position. The resistance sensor can be any suitable sensor made of any suitable materials that allow determining conductance of human tissue being examined.

Further, in an embodiment, tissue receiving area 106 may include a capacitance sensor 118 for detecting whether contact is made between a distal end of a body portion (e.g., a fingertip) and a front end 119 of tissue receiving area 106. In an embodiment, tissue receiving area 106 may be confined by front end 119 and by mechanical leading tracks 108 arranged along the sides of tissue receiving area 106.

It should be noted that while conductivity/resistivity sensor which may be comprised in test pad 116 and/or capacitance sensor 118 are illustrated as being included in their entirety in tissue receiving area 106, this should by no means to be construed as limiting. For example, while tissue receiving area 106 may comprise an electrode that includes a metallic contact, additional components of the conductivity/resistivity sensor may be located outside tissue receiving area 106.

In an embodiment, tissue of a body (e.g., finger) portion may simultaneously engage with conductivity/resistivity sensor 116 and/or capacitance sensor 118 during the emission of light from GT emitter 112 and during detection of emitted light by photodetector 114. For instance, in an operable position, the finger's Hyponychium may engage with capacitance sensor 118, while a medial and lateral tissue portion of a finger portion may be facing GT emitter 112 and photodetector 114, respectively; or vice versa.

It may be imperative that every time a user inserts their finger/tissue into the photodetector 114 it reaches the same test spot and at a certain angle, thus ensuring multi and repetitive tests on the same area of the finger/tissue. In order to achieve this, GM device 100 may include mechanical leader track 108 that leads the tissue portion (e.g., a finger) as close as possible to the same spot each time the tissue portion is placed onto tissue receiving area.

In an embodiment, GM device 100 may include an adaptive element for enabling GM device 100 to accommodate and to fittingly receive fingers or other body portions of various sizes, while ensuring that measurement performed by GM device 100 provide repeatable and consistent outputs, regardless of the size and shape of the body portion received by tissue receiving area 106. In an embodiment, such adaptive element may for example include a mechanical energy storage device 113 and/or 115 (e.g., a compression spring or a pneumatic device) respectively forcing, when in a resting state, GT Emitter 112 and/or photodetector 115 into tissue receiving area 106. A finger portion received by tissue receiving area 106 may thus push GT emitter 112 and/or photodetector 114 outwardly from tissue receiving area 106 (schematically indicated by force vectors F1 and F2, respectively), thus compressing energy storage device 113 and/or 115. In this way, when a finger is operably accommodated in tissue receiving area 106, GT emitter 112 and/or photodetector 114 may be pressed against and, therefore, abut against tissue portions of the finger. For example, GT emitter 112 may be fixed in position relative to the housing of GM device 100, while photodetector 114 is coupled with mechanical energy storage device 115. Once the finger is in operable position, the distance of the finger relative to emitter 112 and photodetector 114 is set and fixed. In an embodiment, mechanical energy storage devices 113 and/or 115 may be coupled with mechanical track leader 108 for adjusting the width of the mechanical track leader in accordance with the size and shape of the finger.

In an embodiment, GM device 100 may further include a processor 122, a memory 124, a user interface 126, and a power source 128 for powering the various components of GM device 100.

Memory 124 may include one or more types of computer-readable storage media. For example, memory 124 may include transactional memory and/or long-term storage memory facilities and may function as file storage, document storage, program storage, and/or as a working memory. The latter may for example be in the form of a static random access memory (SRAM), dynamic random access memory (DRAM), read-only memory (ROM), cache or flash memory. As long-term memory, memory 124, external sensor memory (not shown) may for example include a volatile or non-volatile computer storage medium, a hard disk drive, a solid state drive, a magnetic storage medium, a flash memory and/or other storage facility. A hardware memory facility may for example store a fixed information set (e.g., software code) including, but not limited to, a file, program, application, source code, object code, and the like. As working memory, memory 124 may, for example, process temporally-based instructions.

The various components of GM device 100 may communicate with each other over one or more communication buses (not shown) and/or signal lines (not shown) or wireless signal links (not shown). For example, GM device 100 can be connected wired/wirelessly (such as Bluetooth, Wi-Fi) to processor 122 which include a digital signal processor (DSP), or central processing unit (CPU), micro-controlling unit (MCU), placed or printed circuit board (PCB) on which signal processing software is embedded/installed.

A method, process and/or operation for determining the glucose level in blood may be realized by one or more hardware, software and/or hybrid hardware/software modules. Monitoring engine 150 may be realized by one or more hardware, software and/or hybrid hardware/software modules, e.g., as outlined herein. A module may be a self-contained hardware and/or software component that interfaces with a larger system (Alan Freedman, The Computer Glossary 268, (8^{th} ed. 1998)) and may comprise a machine or machines executable instructions.

For example, a module may be implemented as a controller programmed to, or a hardware circuit comprising, e.g., custom VLSI circuits or gate arrays, off-the-shelf semiconductors such as logic chips, transistors, or other discrete components, configured to cause monitoring device 100 to implement the method, process and/or operation as disclosed herein. A module may also be implemented in programmable hardware devices such as field programmable gate arrays, programmable array logic, programmable logic devices or the like.

For example, memory 124 may include instructions which, when executed processor 122 may result in measuring and monitoring blood glucose level. Such method, process and/or operation may herein be implemented by a “Monitoring Engine” 150 and may be schematically illustrated in FIG. 1 as a block referenced by alphanumeric label “150”. The term “engine” as used herein may also relate to and/or include a module and/or a computerized application. Monitoring engine 150 may cause GT emitter 112 to emit light having a wavelength which lies in the green-turquoise spectrum from GT emitter 112, detect emitted light by photodetector 114, and determine the blood glucose level based on the emitted and detected light, e.g., by determining attenuation and/or absorption of light through tissue accommodated and set in operable position in tissue receiving area 106.

It is noted that the density factor of human tissue may vary as a function of time as a result of changes in, for example, body and/or tissue temperature, body fluids, nutrition, mental pressure, humidity, and/or other factors. Therefore, GM device 100 may include, in an embodiment, an infrared (IR) light emitter 130 operative to emit light, e.g., in the near infrared (NIR) range for determining the relative density of the human tissue when set in operable position. IR light emitter 130 may for example include a laser emitter module or a light emitting diode (LED). The relative density of the human tissue may be taken into account for determining the glucose level in blood. IR light emitter 130 may be operative to emit light having wavelengths and amplitudes suitable for penetrating human tissue at a depth of, e.g., 10 mm or more. Light emitted by IR light emitter may have wavelengths of, for example, about 885 nm, 905 nm, 910 nm or about 940 nm. The amount of light that reaches photodetector 114 may be indicative of the density factor of the human tissue penetrated by the IR light.

It should be noted that terms “amount of light” and “light intensity” as well as grammatical variations thereof may be used interchangeably.

GT emitter 112 and IR emitter 130 may be arranged in various configurations. For example, GT emitter 112 and IR emitter 130 may be combined in a single unit and, e.g., share the same output optics. Alternatively, GT emitter 112 and IR emitter 130 be implemented as separate units. For example, GT emitter 112 and IR emitter 130 may be arranged side-by-side each other. In an embodiment, GT emitter 112 may be sandwiched between two IR emitters 130.

Photodetector 114 comprises one or more detector units, e.g., side-by-side to or opposite of respective light sources for measuring absorption of light by different tissue regions of the same tissue portion received by tissue receiving area 106 in a reflectance or transmittance mode. For example, a plurality of detectors may be juxtaposed to a photo-emitter, i.e., at the same side of the tissue portion, for measuring absorption in the reflectance mode. In another example, a plurality of detectors may be located opposite a photo-emitter for measuring absorption of light in a transmittance mode. The plurality of photodetectors may be operated concurrently or sequentially. Photodetector 114 may be operative to provide an output responsive to incident light at a wavelength ranging, for example, from about 350 nm-1100 nm.

In an embodiment, GT device 100 may include a light concentrator 132 (e.g., concentrating optics) which is positioned between GT emitter 112 and photodetector 114 in relative proximity to photodetector 114 and in alignment with an optical axis of light emitted by GT emitter 112 so that light that propagates through the human tissue is concentrated and focused by the concentrator onto photodetector 114. In other words, when a body portion is set in operable position, light emitted by GT emitter 112 passes through and subsequently emanates from the tissue of the body portion and is then focused by concentrating optics 132 to obtain concentrated or focused light that is incident onto photodetector 114. Concentrator optics 132 may be employed for obtaining increased signal strength responsive to light incident onto photodetector 114 compared to the signal strength that would be obtained if concentrator optics 132 was not obtained.

In some embodiments, GM device 100 may include a plurality of photodetectors 114. In some embodiments, GM device 100 may include a filter (not shown) which may for example be positioned proximate or adjunct to GT emitter 112 so that light first enters filters prior to propagating through tissue positioned in tissue arrangement area 106.

Depending on the arrangement of components of GM device 100, concentrator optics 132 may be placed on top or on the bottom of photodetector 114 relative to the bottom surface of GM device 100

In an embodiment, GM device 100 can derive or determine glucose level in blood by measuring the amount of green-turquoise light reflected back from the tissue. Reflection can be measured through any reflection technology available (direct, relative or others).

In an embodiment, photodetector 114 may operate on, e.g., 100 Hz-1,000 Hz (e.g., 500 Hz) frequency alternating between IR\NIR and GT pulses (starting with IR\NIR pulse) and measure during a blood pulse, for example, the following parameters: (a) absorption of GT in blood, (b) density factor of human tissue through absorption of IR/NIR, and, optionally, (c) applied pressure by the finger onto a capacitance sensor 118 of tissue receiving area 106.

In an embodiment, GM device 100 may comprise a communication module (not shown) which may include one or more I/O device drivers (not shown) and/or network interface drivers (not shown) for enabling the transmission from and/or reception of data at GM device 100. A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G including for example Mobile WIMAX or Long Term Evolution (LTE) advanced, Bluetooth®, ZigBee™, near-field communication (NFC) and/or any other current or future communication network, standard, and/or system.

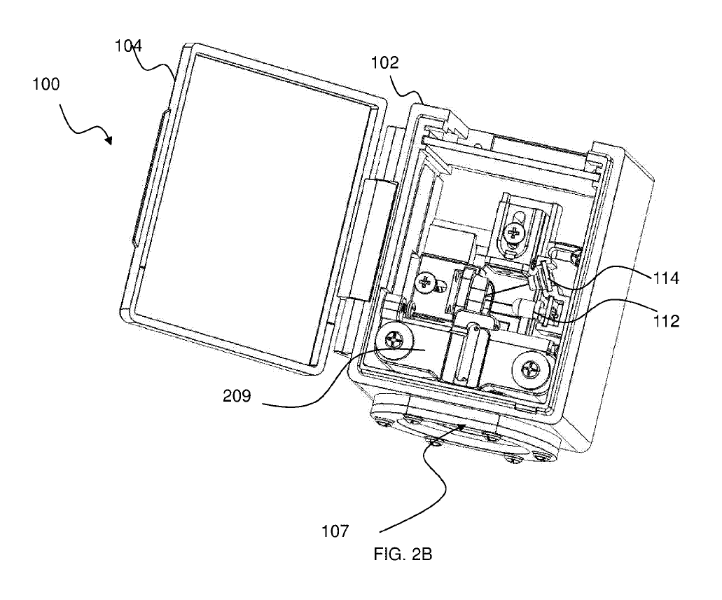

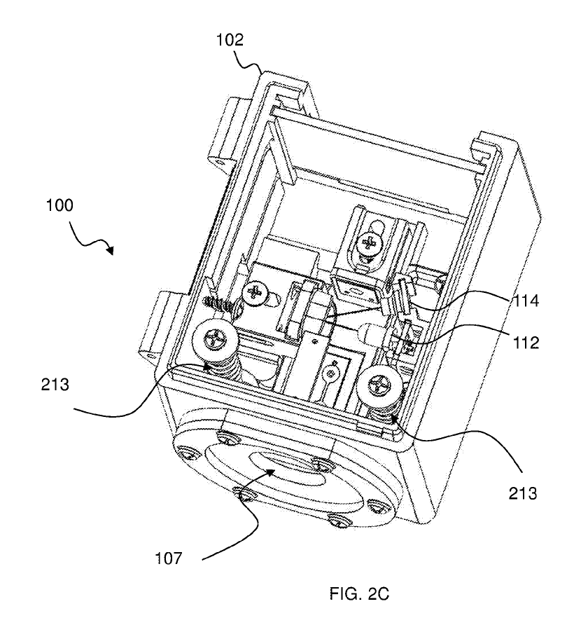

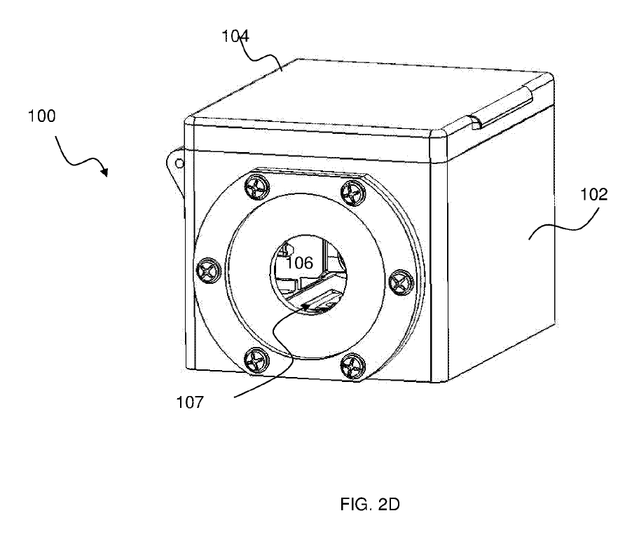

Referring to FIG. 2, monitoring engine 150 may include and/or implement a quality control module 152, a setup module 154, a sampling and processing module 156 and/or an output module 158. Output module 158 may for example include interface 126 which may, for example, include an audio and/or visual display and/or a communication module.

Quality control module 152 may be operative to determine whether the conditions for measuring blood glucose level are met. In an embodiment, quality control module 152 may be employed to validate measurements. Such conditions may relate, for example, to maximal light environment in tissue receiving area 106, existence of human tissue in the reader, normal reader parameters (strength of light, readiness of GT emitter 112, IR emitter 130, photodetector 114, proper operation of capacitance sensor 118, proper operation of the conductivity\electrical resistance sensor), proper emission of light from GT emitter 112 to photodetector 114, and/or proper emission of light from IR emitter 130 to photodetector 114. A corresponding authorizing output may be a precondition to the emission of light by GT emitter 112.

Setup module 154 may be used for adjusting operating parameters of GM device 100. Setup module 154 may for example be used for setting the values of the operating of parameters of, for example, GT emitter 112, IR emitter 130, setting a minimum pressure to be measured by capacitance sensor 118, setting requirements for conductivity/electrical resistance and/or defining calibration curve of green-turquoise light against density factor of human tissue based, e.g., on IR absorbance.

Sampling and processing module 156 may employ processor 112 for executing instructions stored in memory 124 resulting in the sampling and processing of electronic signals provided, e.g., by photodetector 114, which are indicative of the amount of light detected by the photodetector. Following a “green light” from quality control module 152 and setup module 154, sampling and processing module 156 may conduct and manage the blood glucose monitoring through a series of alternate samplings of human tissue density, blood glucose via IR\NIR\GT pulses of light at, e.g., 500 Hz, may process the data collected in real time employing a mathematical algorithm, may compute the relative glucose level in the blood, and may provide an output via output module 106. The output may include the transmission of the data descriptive of the glucose level to another device from output module 106 to another device (not shown) via a communication network (not shown), e.g., for use by a user.

In an embodiment, the glucose level may be determined in the following manner: a human tissue portion (also: test point) is irradiated with GT light by emitter 112. In an embodiment, the GT light may be emitted as a single pulse or a pulse train. At least some of the emitted GT light may pass through the human tissue portion and may be detected by photodetector 114, responsive to which a GT light-based electric signal may be generated. A filter may be employed to obtain data descriptive of noise-reduced detected GT light (e.g., by employing digital signal processing techniques or by employing a filtering circuit). Data descriptive of a magnitude of emitted GT light may be compared with respect to data descriptive of detected GT light to obtain data descriptive of an attenuation of emitted GT light (also referred to as GT “attenuation” data). For instance, a “slope” between emitted and detected GT light may be analyzed to obtain such GT attenuation data. In an embodiment, capacitance sensor data may be obtained for determining whether contact by a tissue portion is made with the sensor. In an embodiment, the amount of pressure to which the tissue portion is subjected to during GT light emission may be determined.

It is noted that the area for which the pressure may be determined may not be exactly the same area through which GT light may propagate but may be located in the vicinity thereof. For example, the pressure which is applied onto a first region of the distal phalanx portion may be determined, while light may enter and a second region of the distal phalanx portion. However, since the surface area of the distal phalanx portion is relatively small so that a pressure application area and a GT light entrance point may be considered to be close to each other relative to the magnitude of the measured pressure, it may be inferred that the pressure is about the same at the GT light entrance point.

In an embodiment, the applied pressure may be taken into account for determining the blood glucose level, as outlined herein below. An increase in pressure applied onto a tissue portion may result in a corresponding increase in the underlying tissue density which, in turn, may alter the amount of blood per a unit tissue volume (also: blood tissue concentration), as compared to the amount of blood per a unit tissue volume if no pressure was applied. A change (increase or decrease) in blood amount per unit tissue volume compared to an initial calibration setup may correspondingly bias a reading in glucose blood concentration towards increased or decreased glucose concentration for the same amount of detected GT light. A forced increase in blood amount per unit volume may thus be taken into account when applying pressure onto the tissue area portion. The corresponding factor may herein be referred to as “tissue density factor”.

The tissue density factor may be determined by assessing a change in the amount of blood per unit volume e.g., in the following manner:

(a) IR light may be emitted by IR emitter 130 towards the tissue portion. The amount of the emitted IR light detected may be converted into IR-based electric signals. A filter may be employed to obtain data descriptive of noise-reduced detected IR light (e.g., by employing digital signal processing techniques or by employing a filtering circuit). Data descriptive of a magnitude of emitted IR light may be compared with respect to data descriptive of detected IR light for obtaining data descriptive of an attenuation of emitted IR light (also: IR attenuation data). For instance, a “slope” between emitted and detected IR light may be analyzed for obtaining IR attenuation data.

The currently obtained IR attenuation data is compared against IR attenuation obtained during calibration setup. If the currently obtained IR attenuation data may differ from the calibration setup IR attenuation data by a certain minimum threshold difference, a corresponding correction for the glucose concentration may be applied. For example, the value of a given blood glucose concentration may be decreased by a multiplicative factor (the tissue concentration factor) to correct for a corresponding increase in measured blood tissue concentration compared to the blood tissue concentration value obtained during calibration setup. Conversely, the value of a given blood glucose concentration may be increased by a multiplicative factor (the tissue concentration factor) to correct for a corresponding decrease in measured blood tissue concentration compared to the blood tissue concentration value obtained during calibration setup.

In an embodiment, IR light may be emitted to determine a time stamp at which the magnitude of a blood pulse was determined to be maximum within a given time window. Sensor data associated with the time stamp may be compared against the calibration setup data. Further, the capacitance sensor signal may be compared with calibration setup data. Based on the maximum blood pulse value compared to the setup data and/or based on the contact signal compared to the setup data, the actual GT signal data may be adjusted (increased, decreased or remain unaltered).

Considering for example the following setup data:

Calibration Measurement 1 (after Fasting and Before Sugar Loading):

A) Freestyle® Blood Glucose concentration measurement: 100 mg/dL.

B) Detected amount of GT light: 55000.

C) Detected amount of IR light: 52000 (e.g., at maximum blood pulse).
The value of detected IR light 52000 for 100 mg/dL is stored by the monitoring engine.

Calibration Measurement 2 (after Fasting and Before Sugar Loading):

D) Freestyle® Blood Glucose concentration measurement: 120 mg/dL.

E) Detected amount of GT light: 53000.

F) Detected amount of IR light: 52000 (e.g., at maximum blood pulse):

The calculated blood glucose concentration for 53000 would thus be about IR IR-52000/52000=1.0 IR factor, GT 55000−53000=2000*1.0=2000, for 120−100=20 mg/dl. 2000/20=100, i.e. 1 mg/dl glucose=100. That is, for every 1 mg/dl glucose change in blood we get a value 100 change in the device.

In an embodiment, it may be assumed that the detected amount of GT light depends on blood glucose concentration. According to such assumed can be not linear dependency, a future actual measurement of detected GT light a blood glucose concentration may form the basis for determining a blood glucose concentration. However, the value of detected GT light of an actual measurement may be adjusted according to an amount of detected IR light, as exemplified herein below.

Actual Measurement (e.g., after Eating):

G) Detected amount of GT light: 51000

H) Detected amount of IR light: 50000
Considering the reduction in IR light, it may be assumed that less amount IR light pass the tissue compared to the calibration measurements.
   The increase is by 2000 units (52000−50000), which corresponds to an increase in “blood concentration” by 4%. Hence, the reading for the amount of GT light on data which is descriptive of the above noted linear (for this sample) dependency between detected GT light and blood glucose concentration has to be corrected by a corresponding 4%, i.e., 55000−51000=4000/100=40, 40*4%=1.6, (40−1.6)+100=138.4 mg/dl.

The actual GT data may be obtained and the relative glucose value may be obtained by comparing the relative value to the setup glucose table in units of mg/dl. Then, data descriptive of the glucose value may then be presented to a user by output module 158 via user interface 126.

Aspects of disclosed embodiments provide for a method of measuring blood glucose in an individual. Most generally, the method includes measuring absorbance of GT light, and measuring or calculating blood glucose based on the GT light absorbed. More specifically, the individual inserts their tissue (such as a finger or ear lobe) in GM device 100 on the test pad 116 and the device is attached to the tissue. Quality control module 152 may determine whether requirements for conducting measurements are met and, if so, provide a corresponding confirmation output responsive to which GT emitter 112 and IR emitter 130 send a pulse of light.

In an embodiment, the terms “distal” end “proximal” refer to the position of a body portion relative to a joint of a limb.

The data obtained from photodetector 114 and, optionally, of conductivity/resistivity sensor 116 and/or capacitance sensor 118 may be processed by processor 122 according to the instructions provided by monitoring engine 150 and displayed to the user via user interface 126 of output module 106.

As already outlined herein, GM device 100 may include a communication module that enables the device to communicably connect with other devices (not shown) and/or services (not shown) via a communication network (not shown). Such other devices and/or services may include a multifunction mobile communication device also known as “smartphone”, a personal computer, a laptop computer, a tablet computer, a server, personal digital assistant, a workstation, a wearable device, a handheld computer, a notebook computer, a vehicular device, a stationary device and/or a home appliances control system. Generally speaking, GM device 100 may be configured to communicably interconnect with other computing devices, systems and services using, for example, the Internet infrastructure. Otherwise stated, GM device 100 may be configured to make use of the “Internet of Things”.

By using GT light blood glucose concentration in the range of, e.g., 70 mg/L to 300 mg/L may be determined. Because the device is non-invasive and easy to use, there is no pricking of the finger or expressing blood needed as with test strips. The measurement is very quick and the glucose measurement reading shows up on the screen within seconds. When used as a continuous monitor, there is no need to replace the device as with sensors, there is improved glucose monitoring, and there can be an alert option in cases of hypoglycemia (low glucose levels) especially during sleep time.

Additional reference is made to FIGS. 2A-2D. In an embodiment, as schematically shown in FIGS. 2A-2C, GT emitter 112 may be positioned with respect to a body portion 205 (e.g., a finger) having a longitudinal axis Z_{finger} so that GT light emitted by GT emitter 112 may have an optical axis Z_{GT} which is about perpendicular to the finger's longitudinal axis Z_{finger}. In an embodiment, IR emitter 114 may be positioned so that IR light emitted by IR emitter may form a non-perpendicular angle with the finger's longitudinal Axis Z_{finger}. In an embodiment, GT emitter 112 and IR emitter 114 may be positioned so that GT light and IR light is incident to body portion 205 substantially at the same point.

FIGS. 2A and 2B schematically shows a bridge element 209 of a mechanical tracking device pressing against finger 205 when in operable position. FIG. 2C schematically shows energy storage devices 213 which press bridge 109 against finger 205.

In the closed configuration shown schematically in FIG. 2D, tissue receiving area 106 may be substantially shielded from ambient light, provided that body portion 205 covers the inlet 107 to the tissue receiving area 106.

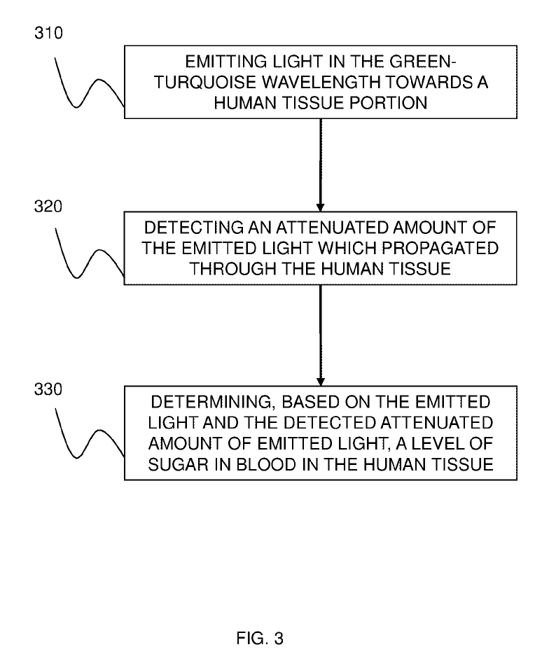

Further referring to FIG. 3, a method for measuring glucose level in blood may include, as indicated by block 310, emitting light in the green-turquoise wavelength towards a human tissue portion.

As indicated by block 320, the method may include detecting an attenuated amount of the emitted light which propagated through the human tissue.

As indicated by block 330, the method may include determining, based on the emitted light and the detected attenuated amount of emitted light, a level of sugar in blood in the human tissue.

Table 1 below shows the test results of three OGTT measurements that were made using the GM device (also: “Biolab™”) and the test results of a measurement that was made using the “Freestyle Freedom-Lite®” Blood Glucose Monitor Device (also: “Freestyle”) of Abbott Diabetes Care Ltd.

TABLE 1

| Measurement 1: | Measurement 2: | Measurement 3: | | |
|---|---|---|---|---|
| GM device | GM device | GM device | | |
| (Biolab) | (Biolab) | (Biolab) | Freestyle: | |
| Maximum | Maximum | Maximum | Blood | |
| emitted light | emitted light: | emitted light | sugar | Approximate |
| 65353: | 65353 | 65353 | level | Time of |
| Detected Light | Detected Light | Detected Light | (mg/dL) | measurement |
| 34521 | 34454 | 34365 | 108 | 10:27 |
| 33532 | 33746 | 33841 | 205 | 11:00 |
| 30226 | 30343 | 30107 | 255 | 11:30 |
| 31934 | 32095 | 31931 | 234 | 12:00 |
| 34449 | 34301 | 33916 | 180 | 12:30 |

Light is indicated as a unit-less numerical value ranging from 0-65353, wherein the value 65353 is indicative of a maximum amount of emitted light. The wavelength of the emitted light was in the range of 495±5 nm. The measurements shown in Table 1 were taken immediately after glucose (loading) intake which followed after 12 hours of fasting. The results were compared to the Freestyle Device. The results of the statistics using SAS are shown below in Table 2:

TABLE 2

| | | | Standard | | | |
|---|---|---|---|---|---|---|
| Variable | N | Mean | Deviation | Median | Minimum | Maximum |
| Average | 5 | 32917 | 1787 | 33706 | 30225 | 34447 |
| Standards | 5 | 144.70114 | 78.92122 | 118.00141 | 78.25812 | 275.14178 |
| Reference | 5 | 196.40000 | 57.02017 | 205.00000 | 108.00000 | 255.00000 |
| Level | | | | | | |

Spearman Correlation Coefficients
N = 5
Prob > |r| under H0: Rho = 0

| | |
|---|---|
| | Reference Level |
| Average | −1.00000 |
| | <.0001 |
| std | 0.10000 |
| | Corr = 0.8729 |

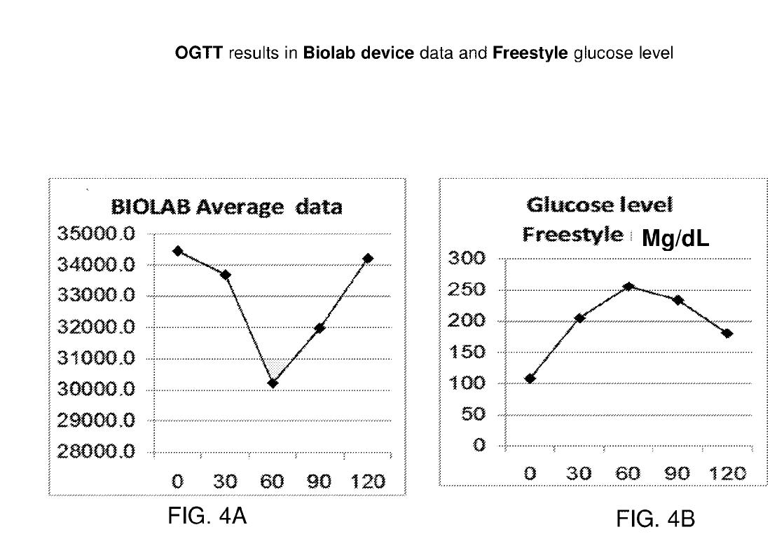

Turning now to FIGS. 4A and 4B, the results of the OGTT measurements are plotted, where in both FIGS. 4A and 4B the X-axis indicates [Minutes]. In FIG. 4B, the Y axis indicates the numerical values indicative of detected amount of green-turquoise light. In FIG. 4B, the Y-axis indicates the amount blood sugar level in units of [mg/dL].

TABLE 3 below also shows the reference signal (the numerical value representing the amount of light detected) which was measured by GM device 100, after fasting and before glucose loading and after glucose loading. The corresponding numerical values obtained by the GM device are also listed in Table 3, as well as their conversion to glucose level. In addition, Table 3 lists the Glucose values obtained using the Freestyle Device after fasting before glucose loading, and after fasting and after glucose loading.

TABLE 3

| | | FreeStyle | | GM Device | |
|---|---|---|---|---|---|
| | | device | GM Device | (Biolab) | |
| | | Glucose | (Biolab) | Signal in 16 bit | |
| | | mg/dl | Glucose | (numerical | |
| | | (control) | mg/dl | value) | Difference % |
| Test | Glucose level after | 94 | 95 | 56742 | 1% |
| 1 | fasting and before | | | | |
| | glucose loading | | | | |
| | Glucose level after | 161 | 168 | 50412 | 1% |
| | glucose loading | | | | |
| Test | Glucose level after | 100 | 101 | 55782 | 5% |
| 2 | fasting and before | | | | |
| | glucose loading | | | | |
| | Glucose after glucose | 151 | 158 | 49880 | 4.50% |
| | loading | | | | |
| Test | Glucose level after | 104 | 100 | 55814 | 3% |
| 3 | fasting and before | | | | |
| | glucose loading | | | | |
| | Glucose after glucose | 195 | 183 | 47424 | 6.50% |
| | loading | | | | |

The embodiments have been described in an illustrative manner, and it is to be understood that the terminology, which has been used is intended to be in the nature of words of description rather than of limitation.

Obviously, many modifications and variations are possible in light of the above teachings. It is, therefore, to be understood that within the scope of the appended claims, the device and method disclosed herein can be practiced otherwise than as specifically described.

In an embodiment, GM device 100 can be used for individual or single measurements of blood glucose (SMBG) and/or for continuous glucose monitoring (CBGM).

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein. For example, any digital computer system can be configured or otherwise programmed to implement a method disclosed herein, and to the extent that a particular digital computer system is configured to implement such a method, it is within the scope and spirit of the disclosure. Once a digital computer system is programmed to perform particular functions pursuant to computer-executable instructions from program software that implements a method disclosed herein, it in effect becomes a special purpose computer particular to an embodiment of the method disclosed herein. The techniques necessary to achieve this are well known to those skilled in the art and thus are not further described herein. The methods and/or processes disclosed herein may be implemented as a computer program product such as, for example, a computer program tangibly embodied in an information carrier, for example, in a non-transitory computer-readable or non-transitory machine-readable storage device and/or in a propagated signal, for execution by or to control the operation of, a data processing apparatus including, for example, one or more programmable processors and/or one or more computers. The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by a communication network.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

“Coupled with” means indirectly or directly “coupled with”.

Where applicable, although state diagrams, flow diagrams or both may be used to describe embodiments, the technique is not limited to those diagrams or to the corresponding descriptions. For example, flow need not move through each illustrated box or state, or in exactly the same order as illustrated and described.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made.

Aspects of the disclosed embodiments are further described in detail by reference to the following experimental examples. These examples are provided for the purpose of illustration only, and are not intended to be limiting unless otherwise specified. Thus, the described embodiments should in no way be construed as being limited to the following examples, but rather, should be construed to encompass any and all variations which become evident as a result of the teaching provided herein.

1-15. (canceled)

16. A device for non-invasively measuring the level of glucose in blood, comprising:
a) a light emitter operative to emit green-turquoise (GT) light in a GT wavelength range incident on a first surface of a human tissue of a given thickness, the GT light transmitted through the human tissue to a second surface;
b) a detector operative to detect the GT light exiting the second surface of the human tissue; and
c) a monitoring engine operative to determine a level of glucose in a blood flow in the human tissue based on a level of absorption of the GT light transmitted through the human tissue.

17. The device of claim 16, wherein the emitted GT light has a wavelength ranging from 490 nm to 505 nm.

18. The device of claim 16, wherein the given thickness is at least 2 mm.

19. The device of claim 18, wherein the given thickness is about 10 mm.

20. The device of claim 16, further comprising an adaptive element configured to apply pressure on the human tissue to increase the blood flow, thereby increasing the ability to record differences in the absorption of GT light.

21. The device of claim 20, wherein the adaptive element includes a mechanical energy storage device.

22. The device of claim 21, wherein the mechanical energy storage device is a compression spring or a pneumatic device.

23. The device of claim 20, wherein the adaptive element is configured to apply pressure on the human tissue to increase the blood flow by abutting the light emitter and/or the detector against the human tissue.

24. The device of claim 16, wherein the human tissue is included in a finger.

25. The device of claim 16, wherein the human tissue is included in an ear lobe.

26. The device of claim 20, further comprising an infrared (IR) light emitter and an IR detector for determining one or both of pressure applied onto the human tissue and occurrence of a maximum blood pulse value in the human tissue.

27. A method for non-invasively measuring the level of glucose in blood, comprising:
a) using a light emitter to emit green-turquoise (GT) light in a GT wavelength range incident on a first surface of a human tissue of a given thickness, the GT light transmitted through the human tissue to a second surface;
b) using a detector to detect the GT light exiting the second surface of the human tissue; and
c) using a monitoring engine to determine a level of glucose in a blood flow in the human tissue based on a level of absorption of the GT light transmitted through the human tissue.

28. The method of claim 27, wherein the emitted GT light has a wavelength ranging from 490 nm to 505 nm.

29. The method of claim 27, wherein the given thickness is at least 2 mm.

30. The method of claim 29, wherein the given thickness is about 10 mm.

31. The method of claim 27, further comprising applying pressure on the human tissue to increase the blood flow, thereby increasing the ability to record differences in the absorption of GT light.

32. The method of claim 31, wherein the applying pressure on the human tissue to increase the blood flow includes applying pressure by abutting the light emitter and/or the detector against the human tissue.

33. The method of claim 27, wherein the human tissue is included in a finger.

34. The method of claim 27, wherein the human tissue is included in an ear lobe.

35. The method of claim 31, further comprising providing an infrared (IR) light emitter and an IR detector determine one or both of pressure applied onto the human tissue and occurrence of a maximum blood pulse value in the human tissue.
